(19) United States
(12) Patent Application Publication
(10) Pub. No.: US 2024/0391858 A1
(43) Pub. Date: Nov. 28, 2024

(54) PRODUCTION, BLENDING AND TRANSPORT OF NET-ZERO-CARBON ORGANIC COMPOUNDS

(71) Applicant: TERRAH2 LLC, WEST LAKE HILLS, TX (US)

(72) Inventors: EVA VINEGAR, WEST LAKE HILLS, TX (US); HAROLD VINEGAR, WEST LAKE HILLS, TX (US)

(21) Appl. No.: 18/671,432

(22) Filed: May 22, 2024

Related U.S. Application Data
(60) Provisional application No. 63/468,409, filed on May 23, 2023.

(51) Int. Cl. C07C 29/151; C07C 29/80

(52) U.S. Cl. CPC C07C 29/1518; C07C 29/80

(57) ABSTRACT

A net-zero-carbon (NZC) methanol or ethanol is produced from a hydrogen gas stored in and subsequently recovered from a geological formation, and from carbon dioxide captured from ambient air. The hydrogen and carbon dioxide are stored and used to produce the NZC methanol and/or ethanol in a synthesis reactor. The NZC methanol and/or ethanol is blended with crude oil and/or condensate at a first location, e.g., at the location of the synthesis reactor, and transported to a second location, e.g., a refiner, where the NZC methanol and/or ethanol are separated from the crude oil and/or condensate. The separated NZC methanol and/or NZC ethanol can subsequently be blended with a gasoline, jet fuel, or diesel to achieve a desired concentration of NZC methanol and/or ethanol in the gasoline or jet fuel or diesel.

### CROSS-REFERENCE TO RELATED APPLICATIONS

This patent application claims the benefit of U.S. Provisional Patent Application No. 63/468,409 filed on May 23, 2023, which is incorporated herein by reference in its entirety.

### FIELD OF THE INVENTION

The present invention relates to the field of sustainable energy and chemicals production, and more specifically, to methods and systems for producing, blending, transporting, and/or distilling net-zero carbon (NZC) methanol and net-zero carbon (NZC) ethanol.

### BACKGROUND OF THE INVENTION

Methanol is an important feedstock to produce various chemicals and materials, as well as a fuel for transportation and power generation. Conventional methanol production methods rely on fossil fuels, which contribute to greenhouse gas emissions and environmental pollution. There is a need for a more sustainable and environmentally friendly approach to produce methanol.

Direct air capture (DAC) methods enable the capture of carbon dioxide (CO₂) from ambient air and provide a potential solution to reduce greenhouse gas emissions. Additionally, renewable energy powered electrolysis or nuclear-powered electrolysis can be employed to generate hydrogen (H₂) from water, further reducing the environmental impact. Excess carbon dioxide and hydrogen may be stored in separate parts of a geological formation and produced as needed to operate the methanol or ethanol synthesis reactor.

Blending methanol with gasoline or jet fuel or diesel can provide benefits such as improved fuel properties, reduced emissions, and increased octane ratings. However, there is a need for an efficient method and system for producing net-zero carbon methanol and/or ethanol and blending it with gasoline or other fuels to maximize the advantages of this approach.

### SUMMARY OF THE INVENTION

According to embodiments, methods and systems are provided for producing net-zero carbon (NZC) methanol. In some embodiments, the methods and systems use net-zero components such as, for example, stored hydrogen and/or carbon dioxide from direct air capture methods. In some embodiments, the NZC methanol and/or NZC ethanol are blended with crude oil and/or condensate onsite. According to embodiments, the methods and systems can include transporting the blended NZC methanol and/or NZC ethanol and crude oil or condensate mixture to a refinery for distillation and further blending and processing.

According to embodiments, the method can comprise some or all of the following steps in any combination and in any order:
1. Capturing carbon dioxide from ambient air using direct air capture methods;
2. Storing the captured carbon dioxide in a geological formation;
3. Producing hydrogen via electrolysis, wherein the electrolysis is powered by renewable energy sources or a zero-carbon energy source such as nuclear power;
4. Storing the produced hydrogen in a geological formation such as a partially depleted, organic-rich, unconventional reservoir, a salt dome, a saline aquifer, or a depleted gas or oil reservoir;
5. Feeding the hydrogen and carbon dioxide to a methanol synthesis reactor;
6. Producing net-zero carbon methanol from the hydrogen and NZC carbon dioxide in the methanol synthesis reactor;
7. Feeding water generated from the carbon dioxide direct air capture method as well as water from the methanol and/or ethanol synthesis reactor back to the electrolyzer;
8. Blending on site the produced NZC methanol and/or NZC ethanol with crude oil and/or condensate from oil and/or condensate wells;
9. Transporting the blended NZC methanol and/or NZC ethanol and crude oil and/or condensate by pipeline, truck, railroad or tanker to a refinery for distillation;
10. Distilling the NZC methanol and/or NZC ethanol from the petroleum fractions of the blend; and
11. Blending the NZC methanol and/or NZC ethanol with gasoline or jet fuel or diesel to achieve a desired ratio of NZC methanol and/or NZC ethanol in gasoline or jet fuel or diesel.

In some embodiments, the system can comprise some or all of the following system components in any combination: a direct air capture unit, a carbon dioxide storage facility, an electrolyzer, e.g., an electrolyzer powered by renewable-energy or other non-polluting or zero-carbon sources, compression and/or pumping equipment in communication with a geological formation such as a partially depleted, organic-rich, unconventional reservoir for storing the hydrogen, a methanol and/or ethanol synthesis reactor, an onsite blending facility for blending the NZC methanol (and/or NZC ethanol) with crude oil and/or condensate, and transportation means, e.g., a pipeline or loading facility, for transporting the NZC methanol and/or NZC ethanol blended with crude oil and/or condensate to a refinery for distillation, a distillation tower for separating the NZC methanol and gasoline or jet fuel or diesel components, a blending unit for mixing the NZC methanol and/or NZC ethanol with gasoline with jet fuel or diesel to achieve the desired blend.

### BRIEF DESCRIPTION OF THE DRAWINGS

FIG. 1 is a schematic representation of a method for producing NZC methanol using stored hydrogen and carbon dioxide from direct air capture methods, in accordance with embodiments of the present invention.

FIG. 2 is a schematic representation of a fractionation distillation tower in which blended crude oil and net-zero carbon methanol are distilled into NZC methanol and petroleum fractions, in accordance with embodiments of the present invention.

FIG. 3 is a schematic representation of a fractionation distillation tower in which blended crude oil and NZC methanol are distilled into NZC methanol and petroleum fractions. The heavier petroleum fractions have residual NZC methanol removed, and the heavier petroleum fractions are catalytically cracked to produce valuable lighter components, such as gasoline, jet fuel, and diesel, in accordance with embodiments of the present invention.

FIGS. 4, 5, 6, 7 and 8 show flowcharts of methods and method steps for producing a net-zero-carbon (NZC) methanol e-fuel or an NZC ethanol e-fuel, in accordance with embodiments of the present invention.

FIGS. 9, 10, 11, 12, 13, 14, 15, 16 and 17 show flowcharts of methods and method steps for synthesizing a net-zero carbon (NZC) organic compound, in accordance with embodiments of the present invention.

FIG. 18 shows a flowchart of a method for producing an automotive fuel with a reduced carbon footprint, in accordance with embodiments of the present invention.

### DETAILED DESCRIPTION OF THE INVENTION

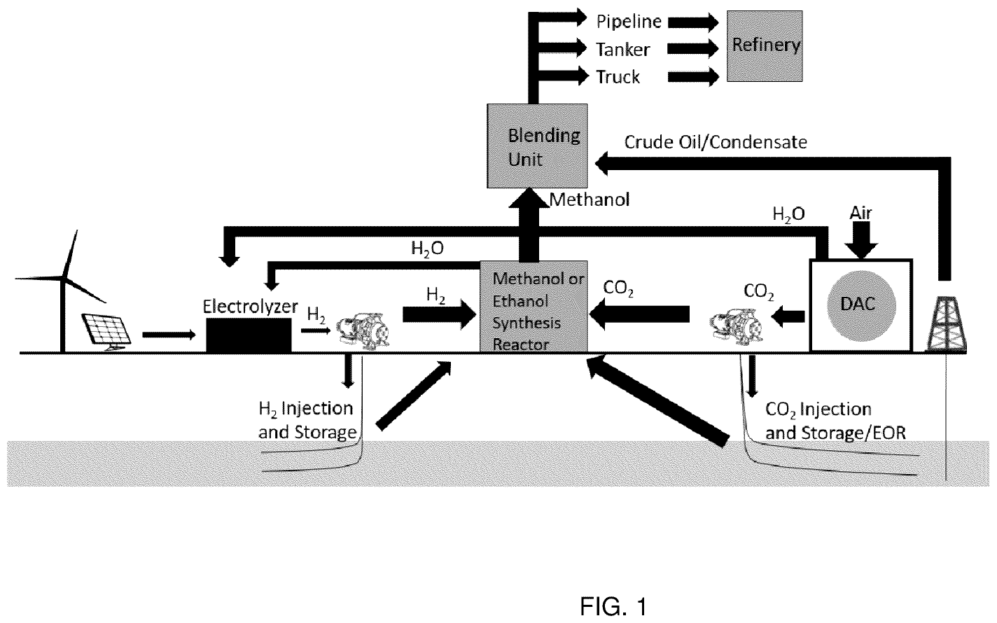

According to embodiments of the present invention as illustrated schematically in FIG. 1, methods and systems are provided for producing net-zero carbon (“NZC”) methanol and/or NZC ethanol. In a non-limiting example, the NZC fuels are produced carbon-neutral or low-carbon components such as stored hydrogen produced using non-polluting, renewable, and/or carbon-neutral energy, and carbon dioxide from direct air capture methods. In some embodiments, the NZC methanol is blended with crude oil, e.g., at or in proximity to an oil production site, and or at or in proximity to the methanol production site. In embodiments, the blended NZC methanol (and/or NZC ethanol) and crude oil (and/or condensate) are transported, e.g., by a pipeline, to a refinery for distillation and further processing.

According to exemplary embodiments, methods are disclosed that include any or all of the following steps in any combination or order of performance:

Step 1: In a non-limiting example, carbon dioxide is captured from ambient air using direct air capture methods, such as neutralization reaction with highly basic KOH solution, pressure-swing adsorption, absorption, or membrane-based systems. The captured carbon dioxide may be stored in suitable storage facilities, such as tanks or underground reservoirs. The direct air capture method is powered using electricity from renewable energy sources, such as solar, wind, hydroelectric power, or from excess hydrogen, or from electricity from nuclear power. In a non-limiting example, in order to achieve economies of scale, the direct air capture unit may remove as much as 0.5 million tonnes/day of atmospheric carbon dioxide.

Step 2: Excess carbon dioxide may be stored in a geological formation such as a partially-depleted organic-rich unconventional reservoir, a salt dome, a saline aquifer, or a depleted gas or oil reservoir. It may be used later to feed the methanol synthesis reactor or in an enhanced oil or natural gas recovery project. Larger direct air capture plants are more economic, so it may be preferable to have excess CO₂ capacity.

Step 3: Hydrogen is produced via electrolysis, e.g., using renewable energy sources, such as solar, wind, or hydroelectric power. The electrolyzer is fed with water, including water generated from the carbon dioxide direct air capture unit and the methanol synthesis reactor, to produce hydrogen and oxygen. Additionally, or alternatively, electrical energy can come from other non-polluting and/or zero carbon sources such as nuclear reactors powered by nuclear fission or nuclear fusion. Additional water for the electrolyzer may come from a water pipeline, a stream, a freshwater aquifer, or trucked or brought in by rail to the site.

Step 4: The produced hydrogen is stored in a geological formation, such as a partially depleted, organic-rich, unconventional reservoir, a salt dome, a depleted gas reservoir, or a saline aquifer. The geological formation acts as a natural hydrogen storage system, providing high storage capacity and reducing the risks associated with above-ground hydrogen storage. Hydrogen can be injected into the geological formation using existing infrastructure, such as wells, pipelines, and compressors.

Step 5: The stored hydrogen and NZC carbon dioxide are fed to a methanol synthesis reactor, where they are combined to produce NZC methanol, as indicated in the equation below. The reactor may utilize a suitable catalyst and operate under specific temperature and pressure conditions to facilitate methanol production.

$${CO}_{2} + 3 {H}_{2} \to C {H}_{3} O H + {H}_{2} O$$

The methanol synthesis reactor may be enlarged in stages to accommodate more methanol production and pipeline transport.

In an alternate non-limiting example, the NZC methanol and CO₂ from direct air capture methods are fed to an ethanol synthesis reactor to produce NZC ethanol. The reactor may utilize a suitable catalyst and operate under specific temperature and pressure conditions to facilitate ethanol production.

In a nonlimiting example, NZC syngas comprised of NZC CO and hydrogen, with additional hydrogen, is used to produce NZC ethanol from NZC methanol. The stochiometric equation is given below.

$$C {H}_{3} O H + ( C O + {H}_{2} ) + {H}_{2} \to C {H}_{3} C {H}_{2} O H + {H}_{2} O$$

where the water is fed to the electrolyzer.

Both NZC methanol and NZC ethanol may be mixed with gasoline or other automotive fuel, e.g., to lower the carbon footprint of the automotive fuel, or they may be used as an electrofuel (“e-fuel”) in advanced automotive engines. Alternatively, some of the ethanol may be derived from agricultural sources.

Step 6: Water generated from the carbon dioxide direct air capture unit and/or from the methanol synthesis reactor may be fed back to the electrolyzer. This integrated use of water from the plant minimizes the amount of additional water required for the electrolyzer.

Step 7: The produced NZC methanol or NZC ethanol is blended onsite with crude oil or condensate using an onsite blending facility. The blending process can be adjusted to achieve desired concentrations and properties of the methanol-crude oil or methanol-condensate mixture (or ethanol-crude oil or ethanol-condensate mixture). The crude oil is first de-watered and de-salted before mixing to avoid corrosion in the pipeline or tanker or truck once mixed with the methanol or ethanol.

As a non-limiting example, blending 2% by volume NZC methanol with a typical light crude oil at the site generates at the refinery a 20% NZC methanol-80% gasoline mixture because the gasoline fraction of the light crude oil is about 10%. This is a M20 Gasoline. The amount blended in with the crude oil may be adjusted at the right ratio at the blending site once the gasoline, jet fuel, or diesel fractions are measured by fractional distillation of the crude oil. Thus, one can have an M10, M20, M30, M50, M85, or even M100 (if not blended) fuel.

Step 8: The blended NZC methanol and/or NZC ethanol and crude oil or condensate mixture is transported by pipeline, truck, or railroad to a refinery for distillation and further processing. Methanol is easier to transport than hydrogen gas in pipelines because methanol is a liquid which can be blended with crude oil, has higher energy density than compressed hydrogen, and can be transported in existing crude oil pipelines. NZC methanol can be decomposed back to hydrogen and CO (i.e., NZC syngas) at the end of the pipeline by catalytic reduction over palladium catalyst. Methanol is also safer to transport than hydrogen gas because leaks are easily detected. If there is a methanol/crude blend spill, it can be easily detected by helicopter. The methanol from a spill rapidly decomposes in the soil or groundwater.

Step 9: At the refinery, the NZC methanol and crude oil or condensate mixture is separated into various fractions, such as NZC methanol, gasoline, and other hydrocarbon products like jet fuel and diesel. The refinery may be configured with multiple distillation columns and distillation trays for co-separating or independently separating methanol and gasoline or jet fuel or diesel fractions. The co-separated or independently separated NZC methanol and gasoline or jet fuel or diesel fractions can be collected and processed for further blending to higher concentrations of NZC methanol in gasoline or jet fuel or diesel, as desired. Alternatively, the NZC methanol can be used as a feedstock to produce hydrogen, chemicals, materials, or fuels. Like NZC methanol, NZC ethanol can be separated from the crude oil or condensate blend in a distillation unit.

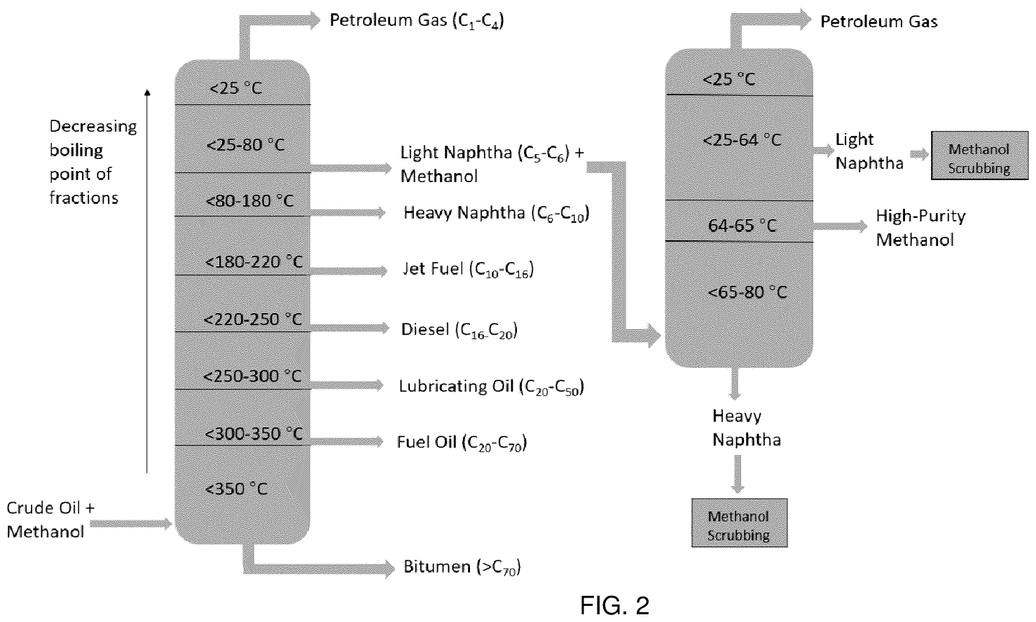

FIG. 2 is a schematic diagram of a fractionation distillation tower in which blended crude oil and NZC methanol can be distilled into NZC methanol and petroleum fractions. A second distillation tower can further separate the gasoline components from the NZC methanol.

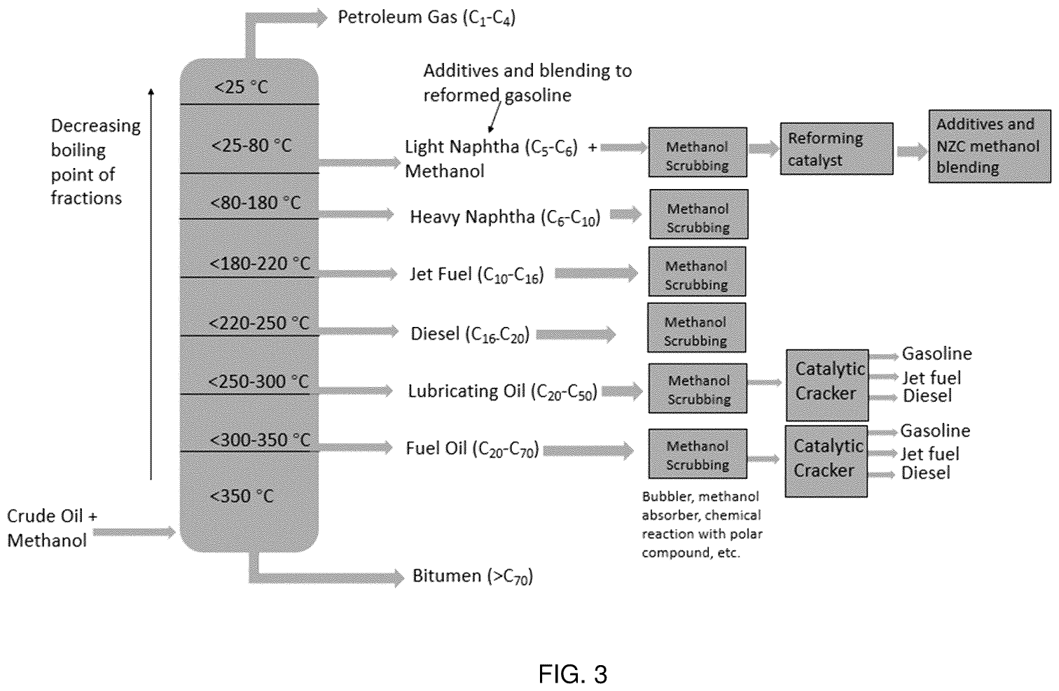

FIG. 3 is a schematic representation of a fractionation distillation tower in which blended crude oil and NZC methanol are distilled into NZC methanol and petroleum fractions. The heavier petroleum fractions have residual NZC methanol removed, and the heavier petroleum fractions are catalytically cracked to produce valuable lighter components, such as gasoline, jet fuel, and diesel, in accordance with embodiments of the present invention. The gasoline may also be reformed by a reforming catalyst before mixing with the NZC methanol and/or NZC ethanol.

Methanol is soluble in gasoline in all proportions, for example M100 (100% methanol), M85 (85% methanol), M15 (15% methanol). Ethanol is also soluble in gasoline in all proportions, for example, E100 (100% ethanol, E85 (85% ethanol, E15 (15% ethanol). Blends of methanol and ethanol can also be prepared, for example, M15E15 (15% methanol, 15% ethanol). Ethanol derived from agricultural sources may also be mixed with the net-zero methanol and blended into gasoline. NZC ethanol may be mixed with gasoline to lower the carbon footprint of the automotive fuel. In another non-limiting embodiment, NZC ethanol is produced from NZC methanol by carbonylation reaction of NZC methanol and NZC carbon monoxide followed by hydrogenation with hydrogen.

In another non-limiting example, NZC syngas comprised of NZC CO and hydrogen, is used to produce NZC methanol and/or NZC ethanol.

In another non-limiting example, NZC methanol may be decomposed to hydrogen and NZC carbon monoxide, and the hydrogen may be used in a vehicle containing a fuel cell.

In another non-limiting example, NZC methanol and/or NZC ethanol may be shipped globally by tanker.

In a non-limiting example, a system comprises some or all of the following components:
a direct air capture unit
a geological formation suited for storing and recovering NZC carbon dioxide,
an electrolyzer powered by renewable or other carbon-neutral energy sources,
a geological formation suited for storing and recovering hydrogen,
a methanol and/or ethanol synthesis reactor,
an onsite blending facility for blending the produced NZC methanol and/or NZC ethanol with crude oil and/or condensate,
transportation means for transporting the blended NZC methanol and/or NZC ethanol and crude oil and/or condensate to a refinery for distillation, a distillation tower for separating the NZC methanol and/or NZC ethanol and gasoline, jet fuel and diesel fractions, and
a blending unit for obtaining a desired ratio of NZC methanol and/or NZC ethanol to gasoline, jet fuel or diesel.

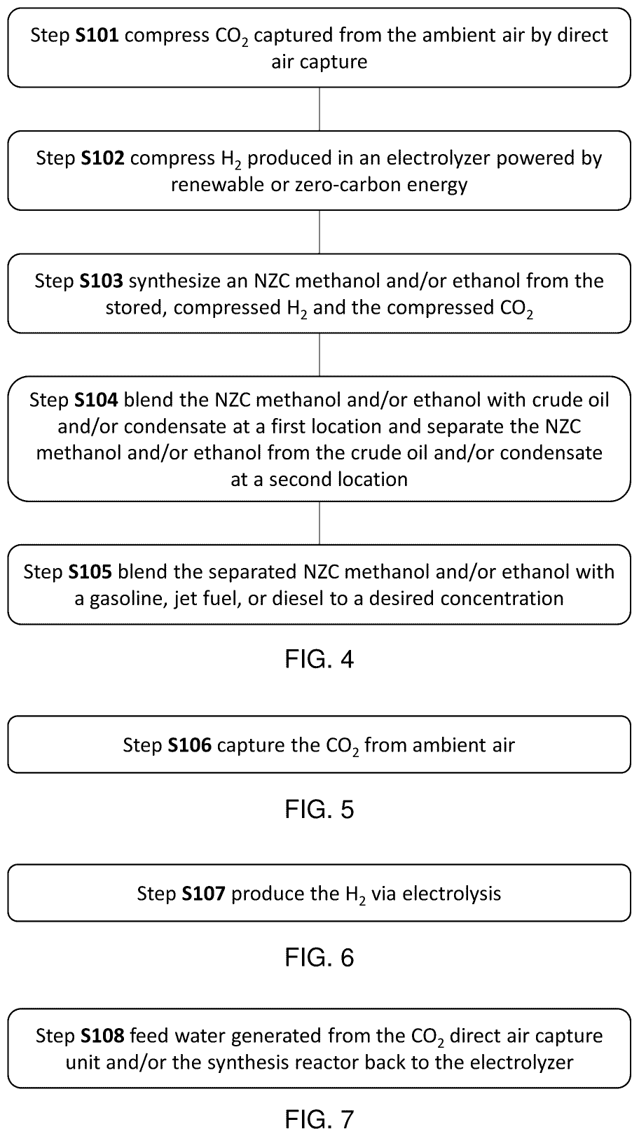

A method is disclosed, according to embodiments, for producing a net-zero-carbon (NZC) methanol e-fuel or an NZC ethanol e-fuel from a hydrogen gas stored in and subsequently recovered from a geological formation, and from carbon dioxide captured from ambient air. As shown in the flowchart of FIG. 4, the method comprises at least Steps S101, S102, S103, S104 and S105.

Step S101 includes compressing the captured carbon dioxide, wherein the capturing of the carbon dioxide from the ambient air is by direct air capture using a renewable and/or zero-carbon energy source.

Step S102 includes compressing hydrogen produced in an electrolyzer, wherein the electrolysis is powered by renewable energy sources and/or zero-carbon energy sources, and storing the hydrogen in a geological formation.

Step S103 includes producing an NZC methanol and/or an NZC ethanol from the stored, compressed hydrogen and the compressed captured dioxide in a synthesis reactor.

Step S104 includes blending the produced NZC methanol and/or NZC ethanol with crude oil and/or condensate at a first location and separating the NZC methanol and/or NZC ethanol from the crude oil and/or condensate at a second location, e.g., after bult transport to the second location. In some embodiments, the first location comprises the synthesis reactor. In some embodiments, the second location comprises a petroleum refinery.

Step S105 includes blending the separated NZC methanol and/or NZC ethanol with a gasoline, jet fuel, or diesel to achieve a desired concentration of NZC methanol and/or NZC ethanol in the gasoline or jet fuel or diesel.

In some embodiments, as shown in the flowchart of FIG. 5, the method additionally comprises Step S106. Step S106 includes capturing the carbon dioxide from ambient air.

In some embodiments, as shown in the flowchart of FIG. 6, the method additionally comprises Step S107. Step S107 includes producing hydrogen via electrolysis.

In some embodiments, as shown in the flowchart of FIG. 7, the method additionally comprises Step S108. Step S108 includes feeding water generated from the carbon-dioxide direct air capture unit and/or the synthesis reactor back to the electrolyzer.

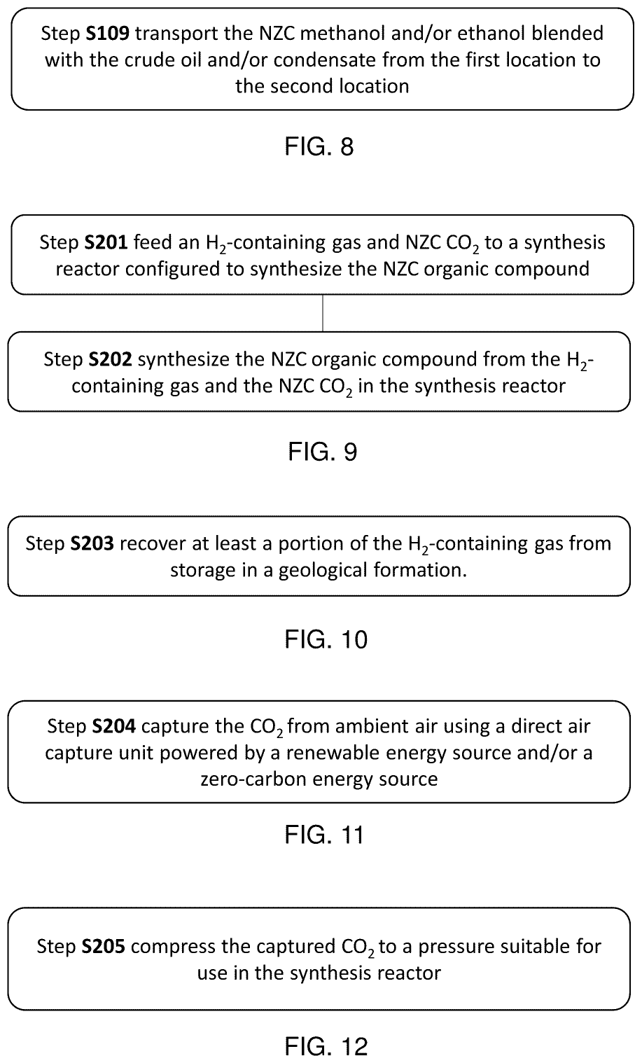

In some embodiments, as shown in the flowchart of FIG. 8, the method additionally comprises Step S109. Step S109 includes transporting the NZC methanol and/or NZC ethanol blended with the crude oil and/or condensate from the first location to the second location.

A method is disclosed, according to embodiments, for synthesizing a net-zero carbon (NZC) organic compound, the NZC organic compound comprising at least one of methanol and ethanol. As shown in the flowchart of FIG. 9, the method comprises at least Steps S201 and S202.

Step S201 includes feeding a hydrogen-containing gas and NZC carbon dioxide to a synthesis reactor configured to synthesize the NZC organic compound. In some embodiments, the hydrogen-containing gas is recovered from storage in a geological formation; the geological formation comprises one of a kerogen-rich, partially depleted unconventional gas reservoir, a salt dome, a saline aquifer, or a depleted gas or oil reservoir.

Step S202 includes synthesizing the NZC organic compound from the hydrogen-containing gas and the NZC carbon dioxide in the synthesis reactor.

In some embodiments, as shown in the flowchart of FIG. 10, the method additionally comprises Step S203. Step S203 includes recovering at least a portion of the hydrogen-containing gas from storage in a geological formation.

In some embodiments, as shown in the flowchart of FIG. 11, the method additionally comprises Step S204. Step S204 includes capturing the carbon dioxide from ambient air using a direct air capture unit, the method powered by a renewable energy source and/or a zero-carbon energy source. In some embodiments, the direct-air capturing of the carbon dioxide is carried out using at least one of: a basic KOH solution reacting with acidic carbon dioxide, pressure-swing absorption-desorption, membrane-based systems, and a cryogenic temperature-based system.

In some embodiments, as shown in the flowchart of FIG. 12, the method additionally comprises Step S205. Step S205 includes compressing the captured carbon dioxide to a pressure suitable for use in the methanol and/or ethanol synthesis reactor.

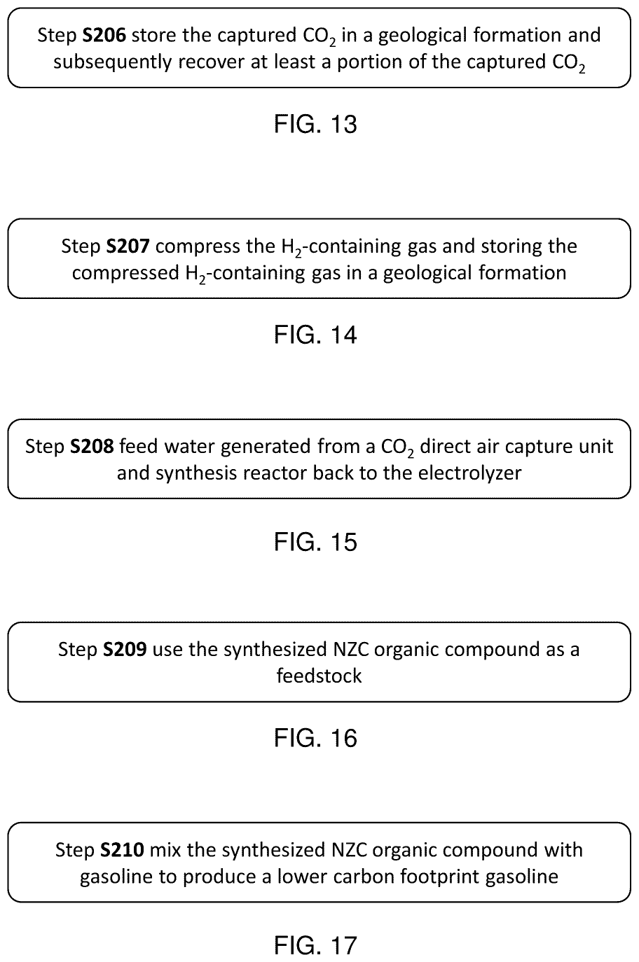

In some embodiments, as shown in the flowchart of FIG. 13, the method additionally comprises Step S206. Step S206 includes storing the captured carbon dioxide in a geological formation and subsequently recovering therefrom at least a portion of the captured carbon dioxide.

In some embodiments, as shown in the flowchart of FIG. 14, the method additionally comprises Step S207. Step S207 includes compressing the hydrogen-containing gas and storing the compressed hydrogen-containing gas in a geological formation.

In some embodiments, as shown in the flowchart of FIG. 15, the method additionally comprises Step S208. Step S208 includes feeding water generated from a carbon dioxide direct air capture unit and synthesis reactor back to an electrolyzer in which the hydrogen-containing gas is produced. In some embodiments, the water fed to the electrolyzer includes water generated from the carbon dioxide direct air capture method or unit and methanol or ethanol synthesis reactor and additional water sources as required.

In some embodiments, as shown in the flowchart of FIG. 16, the method additionally comprises Step S209. Step S209 includes using the synthesized NZC organic compound as a feedstock to produce hydrogen, chemicals, materials, and/or fuels with lower carbon footprint.

In some embodiments, as shown in the flowchart of FIG. 17, the method additionally comprises Step S210. Step S210 includes mixing the synthesized NZC organic compound with gasoline to produce a lower carbon footprint gasoline.

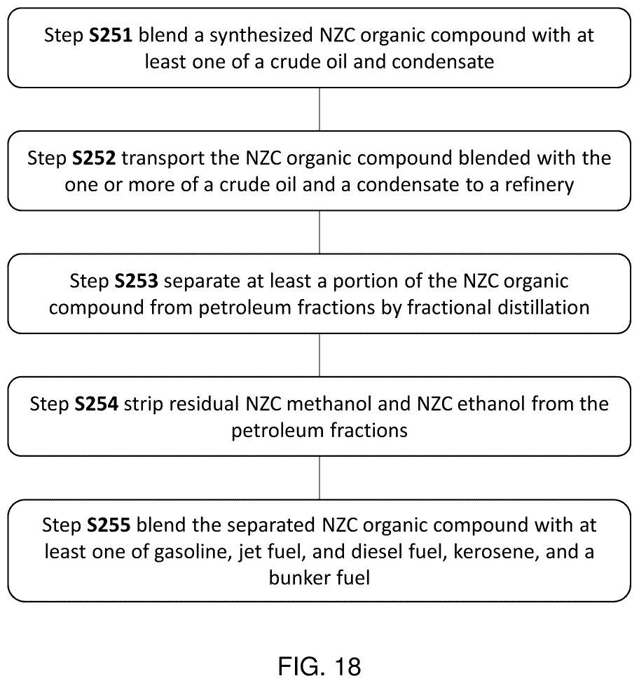

A method is disclosed, according to embodiments, for producing an automotive fuel with a reduced carbon footprint. As shown in the flowchart of FIG. 18, the method comprises at least Steps S251, S252, S253, S254, S255 and S256.

Step S251 includes blending an NZC organic compound synthesized in accordance with Steps S201 and S202, and optionally any one or more of Steps S203, S204, S205, S206, S207, S208, S209, and S210, with at least one of a crude oil and condensate, the synthesized NZC organic compound comprising at least one of methanol and ethanol.

Step S252 includes transporting the NZC organic compound blended with the one or more of a crude oil and a condensate to a refinery. In some embodiments, wherein the refinery comprises a distillation column configured to co-separate the NZC organic compound and gasoline fractions in a distillation tray and, in some such embodiments, the co-separated net NZC organic compound and gasoline fractions are collected and processed for further blending to higher concentrations of NZC methanol and/or NZC ethanol in gasoline. In some embodiments, the refinery comprises a distillation column configured to separate NZC organic compound from the gasoline fractions using separate distillation trays and, in some such embodiments, the separated NZC organic compound and gasoline fractions are collected and processed for further blending to higher concentrations of NZC methanol and/or NZC ethanol in gasoline.

Step S253 includes separating at least a portion of the NZC organic compound from petroleum fractions by fractional distillation at the refinery.

Step S254 includes stripping residual NZC methanol and NZC ethanol from the petroleum fractions.

Step S255 includes blending the separated NZC organic compound with at least one of gasoline, jet fuel, and diesel fuel, kerosene, and a bunker fuel.

In some embodiments, the method results in a blend of a petroleum fuel and an NZC organic compound. In some embodiments, the method results in production of an automotive fuel.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons skilled in the art to which the invention pertains.

1. A method for producing a net-zero-carbon (NZC) methanol e-fuel or an NZC ethanol e-fuel from a hydrogen gas stored in and subsequently recovered from a geological formation, and from carbon dioxide captured from ambient air, the method comprising:
a. compressing the captured carbon dioxide, wherein the capturing of the carbon dioxide from the ambient air is by direct air capture using a renewable and/or zero-carbon energy source;
b. compressing hydrogen produced in an electrolyzer, wherein the electrolysis is powered by renewable energy sources and/or zero-carbon energy sources, and storing the hydrogen in a geological formation;
c. producing an NZC methanol and/or an NZC ethanol from the stored, compressed hydrogen and the compressed captured dioxide in a synthesis reactor;
d. blending the produced NZC methanol and/or NZC ethanol with crude oil and/or condensate at a first location and separating the NZC methanol and/or NZC ethanol from the crude oil and/or condensate at a second location;
e. blending the separated NZC methanol and/or NZC ethanol with a gasoline, jet fuel, or diesel to achieve a desired concentration of NZC methanol and/or NZC ethanol in the gasoline or jet fuel or diesel.

2. The method of claim 1, additionally comprising: capturing the carbon dioxide from ambient air.

3. The method of claim 1, additionally comprising: producing hydrogen via electrolysis.

4. The method of claim 1, additionally comprising: feeding water generated from the carbon-dioxide direct air capture unit and/or the synthesis reactor back to the electrolyzer;

5. The method of claim 1, wherein the first location comprises the synthesis reactor.

6. The method of claim 1, wherein the second location comprises a petroleum refinery.

7. The method of claim 1, additionally comprising: transporting the NZC methanol and/or NZC ethanol blended with the crude oil and/or condensate from the first location to the second location.

8. A method of synthesizing a net-zero carbon (NZC) organic compound, the NZC organic compound comprising at least one of methanol and ethanol, the method comprising:
a. feeding a hydrogen-containing gas and NZC carbon dioxide to a synthesis reactor configured to synthesize the NZC organic compound; and
b. synthesizing the NZC organic compound from the hydrogen-containing gas and the NZC carbon dioxide in the synthesis reactor,
wherein the hydrogen-containing gas is produced by electrolysis using one of a renewable energy source and/or a zero-carbon energy source, and the NZC carbon dioxide comprises atmospheric carbon dioxide directly captured from ambient air.

9. The method of claim 8, wherein the hydrogen-containing gas is recovered from storage in a geological formation, wherein the geological formation comprises one of a kerogen-rich, partially depleted unconventional gas reservoir, a salt dome, a saline aquifer, or a depleted gas or oil reservoir.

10. The method of claim 8, additionally comprising: recovering at least a portion of the hydrogen-containing gas from storage in a geological formation.

11. The method of claim 8, additionally comprising: capturing the carbon dioxide from ambient air using a direct air capture unit, the method powered by a renewable energy source and/or a zero-carbon energy source.

12. The method of claim 8, additionally comprising: compressing the captured carbon dioxide to a pressure suitable for use in the methanol and/or ethanol synthesis reactor.

13. The method claim 8, additionally comprising: storing the captured carbon dioxide in a geological formation and subsequently recovering therefrom at least a portion of the captured carbon dioxide.

14. The method of claim 8, additionally comprising: compressing the hydrogen-containing gas and storing the compressed hydrogen-containing gas in a geological formation.

15. The method of claim 8, additionally comprising: feeding water generated from a carbon dioxide direct air capture unit and synthesis reactor back to an electrolyzer in which the hydrogen-containing gas is produced.

16. The method of claim 8, wherein the direct-air capturing of the carbon dioxide is carried out using at least one of: a basic KOH solution reacting with acidic carbon dioxide, pressure-swing absorption-desorption, membrane-based systems, and a cryogenic temperature-based system.

17. The method of claim 15, wherein the water fed to the electrolyzer includes water generated from the carbon dioxide direct air capture method or unit and methanol or ethanol synthesis reactor and additional water sources as required.

18. The method of claim 8, additionally comprising: using the synthesized NZC organic compound as a feedstock to produce hydrogen, chemicals, materials, and/or fuels with lower carbon footprint.

19. The method of claim 8 additionally comprising: mixing the synthesized NZC organic compound with gasoline to produce a lower carbon footprint gasoline.

20. A NZC organic compound produced in accordance with the method of claim 8.

21. The method of claim 8, wherein the synthesizing includes synthesizing an NZC syngas comprising NZC CO and hydrogen, and producing NZC methanol and/or NZC ethanol from the NCZ syngas.

22. The method of claim 8, wherein the synthesized NZC organic compound includes NZC ethanol produced by carbonylation reaction of NZC methanol and NZC carbon monoxide or an NZC syngas comprising NZC CO and hydrogen, followed by hydrogenation with hydrogen produced from electrolysis powered by renewable energy sources and or zero-carbon energy sources.

23. A method of producing an automotive fuel with a reduced carbon footprint, comprising:
a. blending an NZC organic compound synthesized in accordance with the method of claim 8, with at least one of a crude oil and condensate, the synthesized NZC organic compound comprising at least one of methanol and ethanol;
b. transporting the NZC organic compound, blended with the one or more of a crude oil and a condensate, to a refinery;
c. separating at least a portion of the NZC organic compound from petroleum fractions by fractional distillation at the refinery;
d. stripping residual NZC methanol and NZC ethanol from the petroleum fractions; and
e. blending the separated NZC organic compound with at least one of gasoline, jet fuel, and diesel fuel, kerosene, and a bunker fuel.

24. The method of claim 23, wherein the refinery comprises a distillation column configured to co-separate the NZC organic compound and gasoline fractions in a distillation tray.

25. The method of claim 24, wherein the co-separated net NZC organic compound and gasoline fractions are collected and processed for further blending to higher concentrations of NZC methanol and/or NZC ethanol in gasoline.

26. The method of claim 23, wherein the refinery comprises a distillation column configured to separate NZC organic compound from the gasoline fractions using separate distillation trays.

27. The method of claim 26, wherein the separated NZC organic compound and gasoline fractions are collected and processed for further blending to higher concentrations of NZC methanol and/or NZC ethanol in gasoline.

28. A blend of a petroleum fuel and an NZC organic compound, blended in accordance with the method of claim 23.

29. An automotive fuel produced in accordance with claim 23.

30. A system for producing an automotive fuel, the system comprising:
a. a synthesis reactor for producing an NZC organic compound comprising at least one of NZC methanol and NZC ethanol from a hydrogen-containing gas produced by electrolysis using at least one of a renewable energy source and a zero-carbon energy source such as nuclear fission or nuclear fusion, and carbon dioxide comprising atmospheric carbon dioxide isolated from ambient air; and
b. a blending facility for blending the produced NZC organic compound with crude oil or condensate;
c. transportation means for transporting the blended NZC organic compound and the crude oil or condensate to a refinery comprising a distillation column and a distillation tray.

31. A system for producing and blending an NZC organic compound comprising at least one of methanol and ethanol with crude oil or condensate using geologically stored hydrogen from renewable energy sources or a zero-carbon energy source such as nuclear fission or nuclear fusion and carbon dioxide from direct air capture methods, comprising:
a. a direct air capture unit powered by renewable energy sources or zero carbon energy sources, configured to capture carbon dioxide from ambient air;
b. an electrolyzer powered by renewable energy sources or zero carbon energy sources, configured to produce hydrogen;
c. a compressor and a pump, arranged to store hydrogen in a geological formation and to subsequently recover the hydrogen therefrom;
d. a methanol or ethanol synthesis reactor for producing NZC methanol and/or NZC ethanol from the recovered hydrogen and carbon dioxide; and
e. a blending facility for blending the produced NZC methanol or NZC ethanol with crude oil and/or condensate.

32. The system of claim 31, further comprising, at a distillery, means for receiving blended NZC methanol and/or NZC ethanol and crude oil and/or condensate transported thereto, and a distillation column and one or more distillation trays for co-separating or separately respective NZC organic compound and gasoline fractions.

33. The system of claim 32, wherein the refinery is configured to process the co-separated or separately separated NZC organic compound and gasoline fractions for further blending to higher concentrations of the NZC organic compound in gasoline.

34. The system of claim 31, further comprising a water recirculation system for feeding water generated from the carbon dioxide direct air capture method and/or methanol or ethanol synthesis reactor back to the electrolyzer.
